(19) United States
(12) Patent Application Publication
(10) Pub. No.: US 2007/0185478 A1
(43) Pub. Date: Aug. 9, 2007

(54) Device for local ablation of tissue

(75) Inventor: Phillip J. Cosentino, Gardner, MA (US)

Correspondence Address: Mr.Phillip J. Cosentino, 40 Chestnut Street, Gardner, MA, 01440, US

(73) Assignee: PLASIATHERM, INC., Bedford, NH (US)

(21) Appl. No.: 11/307,452

(22) Filed: Feb. 8, 2006

(51) Int. Cl. A61B 18/04

(52) U.S. Cl. 606/29

(57) ABSTRACT

The invention relates to a catheter device including a coil element comprised of NiCr60 alloy material, whose proximal radial end is coiled about the catheter shaft. The coil element and the thermal containment tube are configured so as to emit thermal energy. This is achieved by a catheter device adapted to deliver thermal energy from a source local to the body tissue to be ablated, adapted for connection to a thermal heat source at its distal end, a fluid conduit for passing a fluid into said ports for anchoring and filling the proximal cuff of the thermal energy device, a separate conduit for placement of necessary connective lines to the thermal energy element and monitoring device and a separate conduit for the drainage of bladder fluids during treatment.

### FIELD OF INVENTION

The invention entails devices for selectively ablating unwanted body tissues, such as excess tissue in the male prostate gland without damaging adjoining tissues.

### BACKGROUND OF THE INVENTION

Benign prostatic hyperplasia, or BPH, commonly referred to as an enlarged prostate, affects more than 50% of men over age 55 and is a worldwide problem. Approximately 200,000 surgeries to treat this condition are presently performed each year in the United States at a cost estimated at $1.6 billion annually. While pharmaceuticals, such as terazosin, may limit prostate growth for a period of time, eventually a surgical solution may be required.

The long-standing surgical procedure for treating BPH is transurethral resection of the prostate or TURP, in which an electrosurgical loop heated by radiofrequency, RF, energy is moved to and from within the prostate to resect, or cut out, troughs of prostate tissue. While a TURP produces satisfactory voiding of urine, it requires general anesthesia and an hour or more of costly operating room time and entails up to 15% impotence, 5 to 10% permanent incontinence, and bleeding requiring a transfusion in up to 10% of the patients. In addition, most TURP patients suffer from retrograde ejaculation, and up to 30% or more of TURP patients experience an infection or other adverse effect.

Recently, high-powered RF roller ball devices have been introduced, which have somewhat reduced the bleeding and other adverse effects of a TURP. However, the use of RF roller ball devices requires general anesthesia and an hour or more of costly operating room time. Holmium lasers can be used for resection of the prostate, producing urine flow results equal to a TURP, while eliminating bleeding and most of the other adverse effects of the above described procedures using RF energy. However, Holmium laser resection typically requires one hour or more of expensive operating room time and general anesthesia.

The interstitial, within tissue, use of microwave, laser, or RF energy to thermally coagulate a portion of the prostate, while taking less time and avoiding general anesthesia, does not significantly reduce the prostate's volume and thus produces less urine voiding relief than a TURP, high power RF roller ball, or Holmium laser resection procedure. In addition, the patients treated with interstitial coagulating devices experience dysuria and discomfort for weeks after the procedure. If the tissue immediately underlying the urethra, which constitutes the exterior surface of the lobes of the prostate, is coagulated, the urethra dies, due to loss of its blood supply, leaving an open, irritating wound. The coagulated tissue then sloughs off and is excreted in the urine over a period of 3-6 weeks.

Laser or RF energy can be used to coagulate a tumor, but coagulation occurs irregularly, as conduction of heat through tissue of differing densities and water content is not uniform. Consequently, it is necessary to closely observe the coagulation procedure to avoid damaging nearby blood vessels, nerves and other vital tissues. While a vaporization zone can be distinguished from normal tissue by ultrasound imaging, coagulated tissue cannot be differentiated from normal tissue by ultrasound imaging. As a result, expensive magnetic resonance imaging, MRI, equipment would be required to visually monitor the coagulation procedure, so that the process can be halted if the coagulation zone approaches important blood vessels, ducts, nerves or other tissues. Unhappily, the use of MRI equipment would increase the cost of an already expensive procedure.

It would be desirable to be able to remove a sufficient amount of prostate tissue to provide immediate voiding and relief of BPH symptoms, while protecting the urethra and the immediately underlying tissue from damage, in a short outpatient procedure, preferably in an outpatient treatment facility or a physician's office under local anesthesia and/or sedation.

### SUMMARY OF THE INVENTION

The present invention provides for the removal of unwanted tissue in a mammalian body, without producing excessive coagulation of surrounding tissues and avoiding thermal damage to a nearby mucosal surface or an adjacent, important blood vessel, duct, nerve or other structure.

This is achieved by a catheter device adapted to deliver thermal energy from a source local to the body tissue to be ablated, adapted for connection to a thermal heat source at its proximal end, a fluid conduit for passing a fluid into ports for anchoring and filling the proximal cuff of the thermal energy device, a separate conduit for placement of necessary connective lines to the thermal energy element and monitoring device, and a separate conduit for the drainage of bladder fluids during treatment.

The fluid can be passed through the ports by positive pressure, and can be withdrawn by vacuum, i.e., negative pressure. The distal end of the flexible energy conduit is adapted to emit energy to a predetermined tissue site so as to ablate the tissue.

In one embodiment of the present invention, energy, such as thermal energy, is transmitted through a coil element comprised of NiCr60 alloy material, whose proximal radial end is coiled about the catheter shaft, encased within a silicone or comparable tube forming an enclosed cuff about the catheter shaft. Encasing the thermal coil in a tube provides a significant holding vessel for liquid to be heated and utilized as a thermal heat transfer media to the prostate wall. A second tube, cuff, is attached directly over the coil tube and is utilized in the placement of a temperature sensing device within the boundary layer between the two tube materials, allowing measurement and subsequent control of the thermal heat energy at the point of contact with the prostate gland via a control device exterior of the subject.

In use, the present invention is inserted into tissue and oriented to produce thermal energy in a desired pattern, away from a region or tissue to be preserved, such as the mucosa or endothelial surface of an organ or an important blood vessel, duct, nerve or other structure, to prevent thermal damage thereto. The device can be orientated in a lineal direction while delivering thermal energy, or advanced and/or withdrawn while delivering thermal energy, or both. Such a device, for example, could be used to ablate a portion of the lobes of the prostate, without damaging the sensitive urethra, or its immediately underlying, supportive tissue, or without damaging surrounding normal tissue or a nearby major blood vessel, duct, nerve or other structure.

### BRIEF DESCRIPTION OF THE DRAWINGS

Other objects, features and advantages will occur to those skilled in the art from the following description of the preferred embodiment, and the accompanying drawings, wherein corresponding reference characters indicate corresponding members in which:

FIG. 1 is a vertical sectional view of a male pelvic region showing the urinary organs affected by benign prostatic hyperplasia.

FIG. 2 is a diagram illustrating the thermal therapy catheter of the present invention.

FIG. 3 is a sectional view of the thermal therapy catheter heating element and temperature sensor of the present invention.

FIG. 4 is a cross-sectional view of a portion of the thermal therapy catheter of the present invention in the vicinity of a dual-layered balloon and anchor balloon illustrating internal lumens.

FIG. 5 is a distal end view of the thermal therapy catheter of the present invention, illustrating connections to various lumens.

FIG. 6 is a proximal view of the thermal therapy catheter of the present invention, illustrating the proximal balloons.

### DESCRIPTION OF THE PREFERRED EMBODIMENTS OF THE INVENTION

While this invention is susceptible of embodiment in many different forms, specific embodiments are shown in the drawings and are described herein in detail, with the understanding that the present disclosure is to be considered as an exemplification of the principles of the invention and is not to be limited to the specific embodiments illustrated.

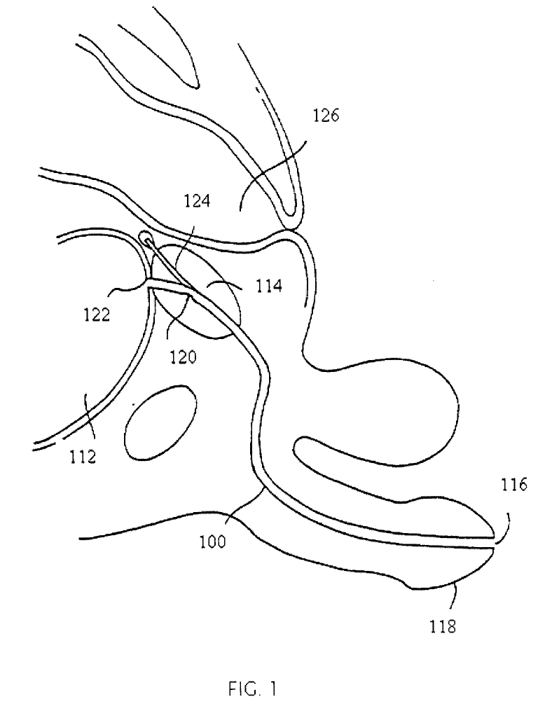

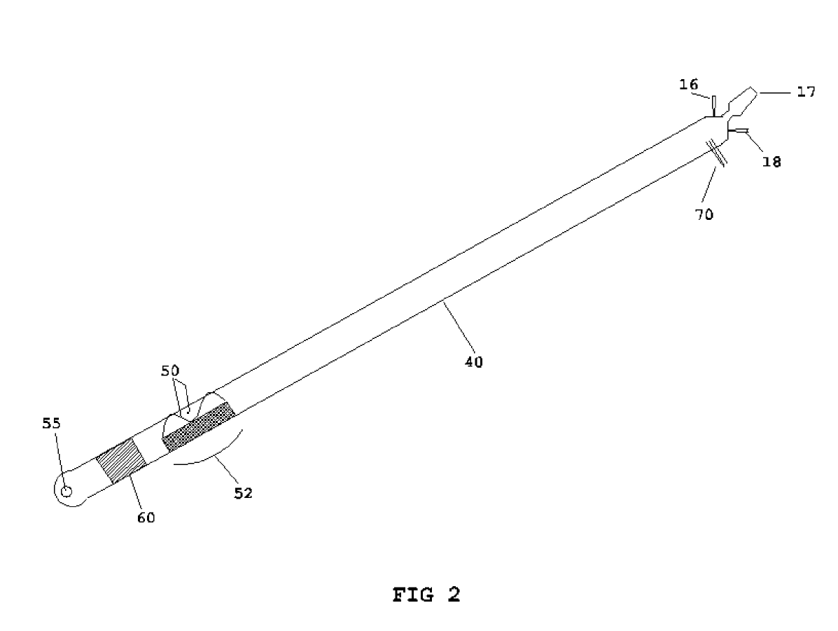

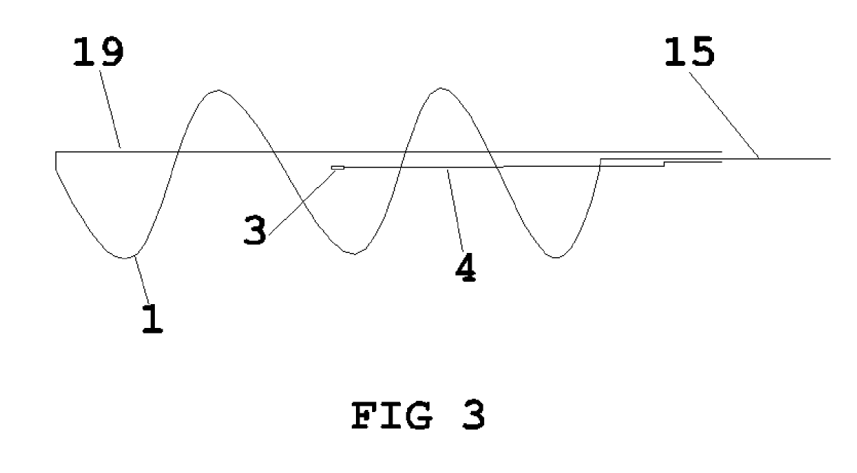

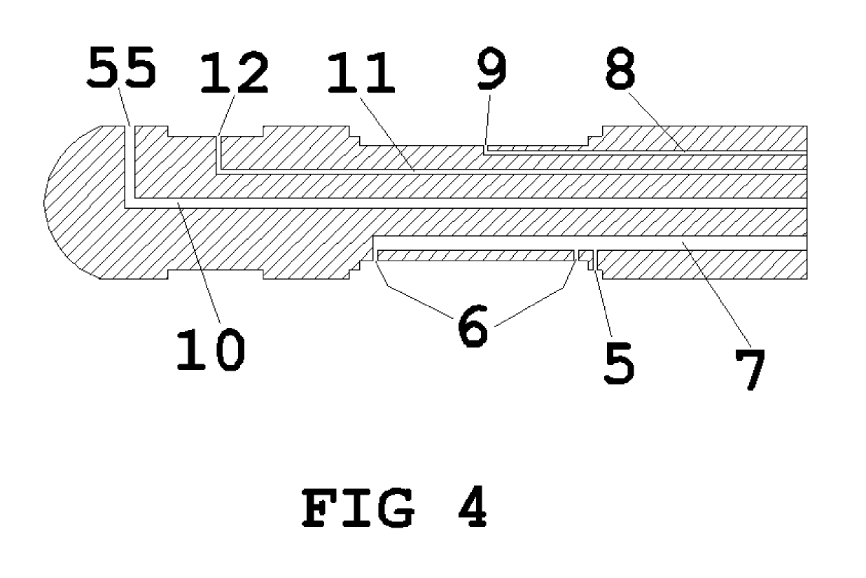

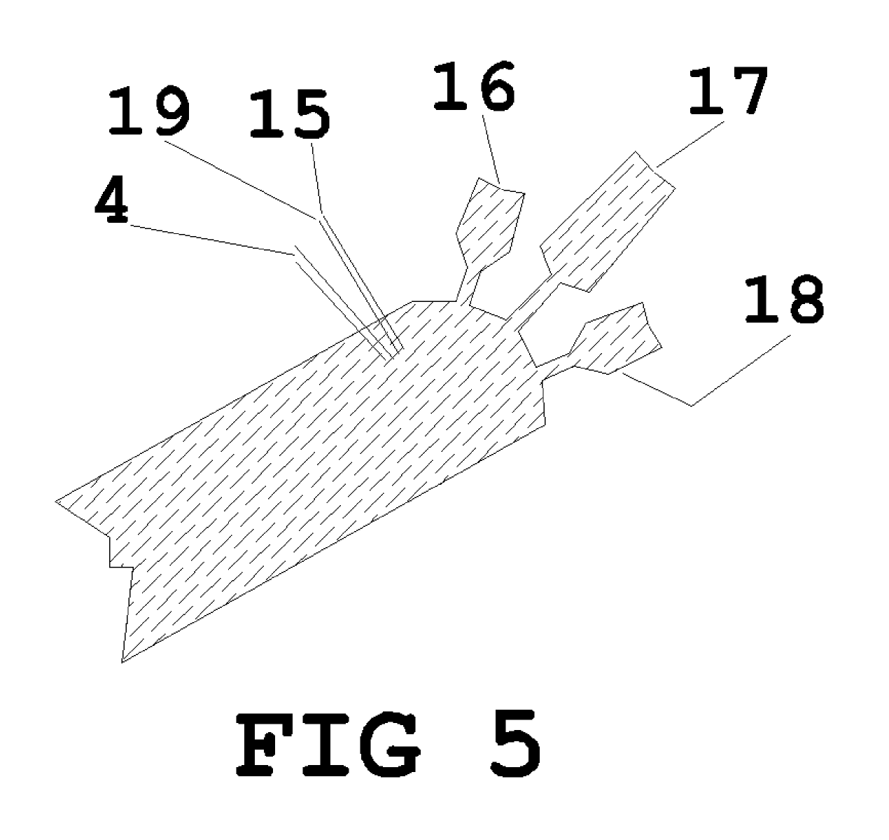

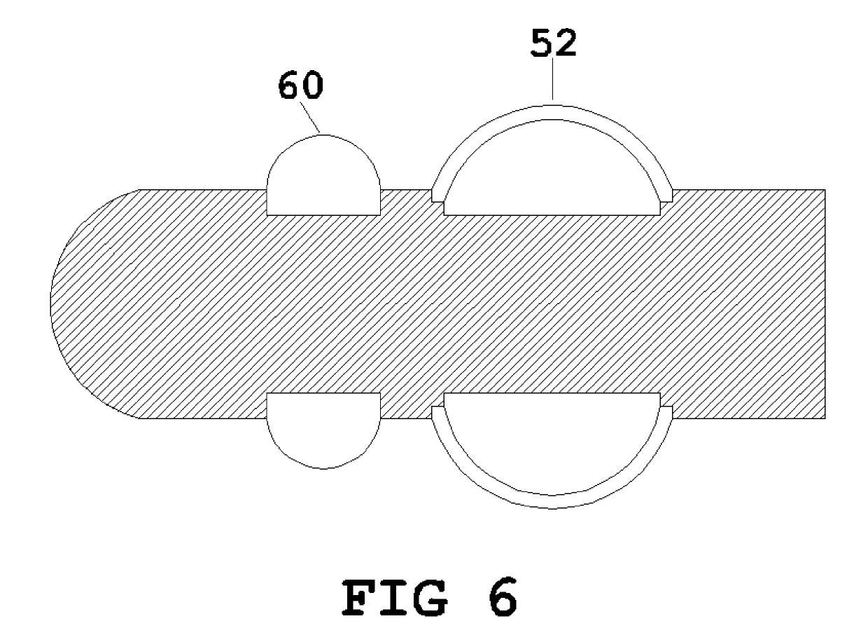

FIG. 1 is a vertical sectional view of a male pelvic region showing the effect benign prostatic hyperplasia, BPH, has on the urinary organs. Urethra 100 is a duct leading from bladder 112, through prostate 114 and out orifice 116 of penis end 118. Benign tumorous tissue growth within prostate 114 around urethra 100 causes constriction 120 of urethra 100, which interrupts the flow of urine from bladder 112 to orifice 116. Heating and necrosing the encroaching tumorous tissue can effectively remove the tumorous tissue of prostate 114, which encroaches urethra 100 and causes constriction 120. Ideally, with the present invention, a selected volume of tissue of prostate 114 can be necrosed while preserving the tissue of urethra 100 and adjacent tissue such as ejaculatory duct 124 and rectum 126. This is achieved by thermal therapy catheter, a preferred embodiment of the present invention, which is shown in FIG. 2, FIG. 3, FIG. 4, FIG. 5, and FIG. 6.

FIG. 2 is a diagram illustrating an embodiment of a thermal therapy catheter system of the present invention. This system comprises multi-lumen shaft 40, fluid connection manifolds 16, 17 and 18, heat element and temperature monitoring elements 50, dual-layered balloon 52, urine drainage port 55, anchor balloon 60, and heat element and temperature monitoring connections 70. The catheter material is composed of a thermoplastic elastomer or a similar material.

FIG. 3 is a diagram illustrating a heating element and temperature sensor system of the present invention. This system comprises a heating element 1,54 mm in length with a diameter of 0.2032 mm constructed of NiCr60 alloy resistance wire coiled about the catheter shaft 40, connected to a length of insulated 32awg copper wire 19 at the proximal end and a length of insulated 32awg copper wire 15 at the distal end of heating element 1, also comprising a negative temperature coefficient, NTC, thermistor 3 connected to two insulated 42awg copper conductors 4 which is inserted between the two layers of the dual-layered balloon 52. Other embodiments could use other materials and devices for the heating element and temperature sensor.

FIG. 4 is a diagram illustrating one embodiment of the lumens and ports on the proximal end of a catheter system of the present invention. This system comprises a temperature sensor conductor port 5 for the passage of temperature sensor conductors within conductor lumen 7, further comprising heating element conductor ports 6 for the passage of heating element conductors within the conductor lumen 7, further comprising a dual-layered balloon fluid port 9 for the introduction of fluid to inflate the dual-layered balloon 52 in direct fluid path with dual-layered balloon fluid lumen 8, further comprising an anchor fluid port 12 for the introduction of fluid to inflate anchor balloon 60 in direct fluid path of anchor balloon fluid lumen 11 and further comprising a urine drainage port 55 for drainage of bladder fluid in direct fluid path of urine drainage lumen 10. Further, the ports 5 and 6 through which the temperature sensor and heating element conductors pass are sealed with a suitable potting material to prevent fluid from leaking from the fluid lumen 8 into the conductor lumen 7.

FIG. 5 is a diagram illustrating one embodiment of lumen connectors and conductor ends of a catheter system of the present invention. This system comprises a set of temperature sensor conductors 4 for terminal connection to a control device separate of this invention and system, further comprising a distal heating element conductor 15 for terminal connection to a control device separate of this invention and system, further comprising a proximal heating element conductor 19 for terminal connection to a control device separate of this invention and system, further comprising a dual-layered balloon inflation connector 16 for the introduction of fluid to dual-layered balloon 52 being in direct fluid path with dual-layered balloon fluid lumen 8 also being in direct fluid path with dual-layered balloon fluid port 9, further comprising a anchor balloon inflation connector 18 for the introduction of fluid to anchor balloon 60 being in direct fluid path with anchor balloon fluid lumen 11 also being in direct fluid path with dual-layered balloon fluid port 12, and comprising a urine drainage connector 17 for the drainage of bladder fluid in direct fluid path of urine drainage lumen 1 0 also being in direct fluid path with urine drainage port 55.

FIG. 6 is a diagram of one-embodiment illustrating balloon placements at the proximal end of a catheter system of the present invention. This system comprises an anchor balloon 60 for bladder anchorage in the bladder at the bladder neck, further comprising a dual-layered balloon 52 for retention of fluid to be heated for treatment of prostate tissue.

Referring again to FIG. 2, shaft 40 is connected to a control device separate of this invention and system. In this embodiment, shaft 40 is a multi-lumen, Foley-type urethral catheter, with inflatable retention balloon 60 at proximal end. Shaft 40, which has an outer diameter of about 18 French, 6 millimeters, is generally circular in cross-section, and is both long enough and flexible enough to permit insertion of proximal shaft end into the penis orifice 116, through the urethra 100 into bladder 112 where retention balloon 60 is inflated and seated against the bladder neck 122 to secure the catheter in place. This enables precise location of heating and sensing elements 50 with respect to prostate tissue.

Thermal welding or a comparable attachment technique such as adhesive bonding, at one or more points on the outer surface of shaft 40 around heating element 1, attaches dual-layered balloon 52 to the outer surface of shaft 40 near proximal end, preferably. Dual-layered balloon 52 is wrapped around shaft 40. The construction and operation of dual-layered balloon 52 is described in more detail below.

In this embodiment dual-layered balloon 52, wherein the dual-layered balloon 52 is constructed of one balloon around the outer surface of the catheter shaft 40 and a balloon around the outer surface of the balloon around the outer surface of the catheter shaft forming dual-layered balloon 52, attached to catheter shaft 40 by thermal welding or a comparable attachment technique such as adhesive bonding.

An apparatus aspect of the present invention is a medical catheter device for delivering localized energy to a tissue in a patient's body in an amount sufficient to ablate or vaporize the tissue. In use, the catheter device is suitably positioned within a patient's body by insertion through a body lumen, cavity or surgically created passageway, and advanced to a predetermined site within the body. The device of the present invention is particularly suited for the vaporization of prostate tissue and involves the use of thermal energy.

Numerous variations and modifications of the embodiments described above may be effected without departing from the spirit and scope of the novel features of the invention. No limitation with respect to the specific apparatus illustrated herein is intended or should be inferred. The above description is, of course, intended to cover by the appended claims all such modifications as fall within the scope of the claims.

What is claimed is:

1. A device for treating tissue adjacent to a body lumen, said device comprising a catheter shaft insertable into the body lumen; a heating element coiled around and carried by the said catheter shaft; a dual-layered balloon around the said heating element around the said_catheter shaft, opposing ends of the said dual-layered balloon being sealingly connected to the said catheter shaft to form a fluid chamber between the said dual-layered balloon and the said_catheter shaft and a sensor chamber between the balloon layers of the said dual-layered balloon and a fluid lumen for delivery of a fluid in the said catheter shaft and the said fluid chamber in a defined fluid flow path to inflate the said dual-layered balloon into contact with the wall of the said body lumen.

2. The device of claim 1, wherein the said dual-layered balloon is substantially distensible and is inflatable to a predetermined inflated diameter.

3. The device of claim 2, wherein the said dual-layered balloon is composed of a silicone material, and wherein the predetermined inflated diameter is set by inflating the said dual-layered balloon to the said predetermined inflated diameter by introducing a prescribed amount of fluid.

4. The device of claim 1, wherein the said fluid delivery lumen comprises a lumen in the catheter shaft being in fluid communication with the said dual-layered balloon.

5. The device of claim 4, wherein the said dual-layered balloon comprises one balloon around the outer surface of the said catheter shaft and a balloon around the outer surface of the said balloon around the outer surface of the said catheter shaft.

6. The device of claim 1, further comprising a temperature sensor located between the said dual-layered balloons surrounding the outer surface of the said catheter shaft, and a lumen in the said catheter shaft allowing placement of the said temperature sensor and temperature sensor conductors, with the said temperature sensor placed so as to sense a temperature of body tissue adjacent to the outer balloon surface in proximity to the said temperature sensor.

7. The device of claim 6, further comprising a heating element coiled about the said catheter shaft located adjacent to the said dual-layered balloon, and a lumen in the said catheter shaft allowing the placement of conductors for connection to the said heating element.

8. The device of claim 1, wherein the said catheter shaft is insertable into a urethra for treating prostate tissue adjacent the urethra.

9. The device of claim 1, further comprising a urine drainage lumen in the said catheter shaft.

10. The device of claim 1, further comprising a balloon inflation lumen in the said catheter shaft, and a retention balloon at the end of the catheter shaft, the said retention balloon being in fluid communication with the said balloon inflation lumen so as to be inflatable in a bladder to secure the said catheter shaft in place in the urethra.

11. The device of claim 1, further comprising a electrical plug connected to a distal end of the catheter shaft, the plug comprising a first piece having at least four pins thereon; wherein the said first piece when assembled forms a means to connect to a controlling device.

12. The device of claim 1, wherein the said heating element comprises a nickel chromium alloy material having a plurality of windings.

13. The device of claim 12, wherein the said windings of the said heating element have a pitch that is consistent along a length of the said catheter shaft.
